(19) United States
(12) Patent Application Publication
(10) Pub. No.: US 2010/0204570 A1
(43) Pub. Date: Aug. 12, 2010

(54) ANCHOR MARKERS

(76) Inventor: Paul Lubock, Laguna Niguel, CA (US)

Correspondence Address: EDWARD J. LYNCH, PATENT ATTORNEY, FOUR EMBARCADERO CENTER, SUITE 1700, SAN FRANCISCO, CA, 94111, US

(21) Appl. No.: 12/701,448

(22) Filed: Feb. 5, 2010

Related U.S. Application Data
(60) Provisional application No. 61/206,974, filed on Feb. 6, 2009.

(51) Int. Cl. A61B 5/05

(52) U.S. Cl. 600/426

(57) ABSTRACT

The invention is directed to an anchor marker that is secured to adjacent tissue in order to prevent or minimize displacement of the marker and a method of delivering such a marker to a patient's body cavity such as a cavity in a patient's breast after a biopsy or lumpectomy. The anchor marker has an anchor element and a marker element which is secured to the anchor element. The anchor element penetrates into surrounding tissue and secures the marker from movement. The marker element is remotely imagable by ultrasound, x-ray, MRI and the like and preferably has incorporated imagable material to facilitate such imaging.

### RELATED APPLICATIONS

This application is related to and claims priority from provisional application Ser. No. 60/206,974 filed on Feb. 6, 2009, which is incorporated herein by reference in its entirety.

### FIELD OF THE INVENTION

The invention is generally directed to remotely detectable, intracorporeal biopsy site markers and devices.

### BACKGROUND OF THE INVENTION

In diagnosing and treating certain medical conditions, it is often desirable to mark a suspicious body site for the subsequent taking of a biopsy specimen, for delivery of medicine, radiation, or other treatment, for the relocation of a site from which a biopsy specimen was taken, or at which some other procedure was performed. As is known, obtaining a tissue sample by biopsy and the subsequent examination are typically employed in the diagnosis of cancers and other malignant tumors, or to confirm that a suspected lesion or tumor is not malignant. The information obtained from these diagnostic tests and/or examinations is frequently used to devise a therapeutic plan for the appropriate surgical procedure or other course of treatment.

In many instances, the suspicious tissue to be sampled is located in a subcutaneous site, such as inside a human breast. To minimize surgical intrusion into a patient's body, it is often desirable to insert a biopsy instrument into the body for extracting the biopsy specimen while imaging the procedure using fluoroscopy, ultrasonic imaging, x-rays, magnetic resonance imaging (MRI) or any other suitable form of imaging technique or palpation. Examination of tissue samples taken by biopsy is of particular significance in the diagnosis and treatment of breast cancer. In the ensuing discussion, the biopsy and treatment site described will generally be the human breast, although the invention is suitable for marking sites in other parts of the human and other mammalian body as well.

Periodic physical examination of the breasts and mammography are important for early detection of potentially cancerous lesions. In mammography, the breast is compressed between two plates while specialized x-ray images are taken. If an abnormal mass in the breast is found by physical examination or mammography, ultrasound may be used to determine whether the mass is a solid tumor or a fluid-filled cyst. Solid masses are usually subjected to some type of tissue biopsy to determine if the mass is cancerous.

If a solid mass or lesion is large enough to be palpable, a tissue specimen can be removed from the mass by a variety of techniques, including but not limited to open surgical biopsy, a technique known as Fine Needle Aspiration Biopsy (FNAB) and instruments characterized as “vacuum assisted large core biopsy devices”.

If a solid mass of the breast is small and non-palpable (e.g., the type typically discovered through mammography), a vacuum assisted large core biopsy procedure is usually used. In performing a stereotactic biopsy of a breast, the patient lies on a special biopsy table with her breast compressed between the plates of a mammography apparatus and two separate x-rays or digital video views are taken from two different points of view. A computer calculates the exact position of the lesion as well as depth of the lesion within the breast. Thereafter, a mechanical stereotactic apparatus is programmed with the coordinates and depth information calculated by the computer, and such apparatus is used to precisely advance the biopsy needle into the small lesion. The stereotactic technique may be used to obtain histologic specimens. Usually at least five separate biopsy specimens are obtained from locations around the small lesion as well as one from the center of the lesion.

The available treatment options for cancerous lesions of the breast include various degrees of mastectomy or lumpectomy, radiation therapy, chemotherapy and combinations of these treatments. However, radiographically visible tissue features, originally observed in a mammogram, may be removed, altered or obscured by the biopsy procedure, and may heal or otherwise become altered following the biopsy. In order for the surgeon or radiation oncologist to direct surgical or radiation treatment to the precise location of the breast lesion several days or weeks after the biopsy procedure was performed, it is desirable that a biopsy site marker be placed in the patient's body to serve as a landmark for subsequent location of the lesion site.

There are a number of biopsy probes and delivery devices that are presently used to place biopsy site markers within the body. A biopsy site marker may be a permanent marker (e.g., a metal marker visible under x-ray examination), or a temporary marker (e.g., a bioabsorbable marker detectable with ultrasound). While current radiographic type markers may persist at the biopsy site, an additional mammography generally is performed at the time of follow up treatment or surgery in order to locate the site of the previous surgery or biopsy.

As an alternative or adjunct to radiographic imaging, ultrasonic imaging (herein abbreviated as “USI”) or visualization techniques can be used to image the tissue of interest at the site of interest during a surgical or biopsy procedure or follow-up procedure. USI is capable of providing precise location and imaging of suspicious tissue, surrounding tissue and biopsy instruments within the patient's body during a procedure. Such imaging facilitates accurate and controllable removal or sampling of the suspicious tissue so as to minimize trauma to surrounding healthy tissue.

For example, during a breast biopsy procedure, the biopsy device is often imaged with USI while the device is being inserted into the patient's breast and activated to remove a sample of suspicious breast tissue. As USI is often used to image tissue during follow-up treatment, it may be desirable to have a marker, similar to the radiographic markers discussed above, which can be placed in a patient's body at the site of a surgical procedure and which are visible using USI. Such a marker enables a follow-up procedure to be performed without the need for traditional radiographic mammography.

Unfortunately it is possible for an implanted biopsy site marker to change location or shift in relation to the site of the previous procedure. Current biopsy markers are known to migrate for a variety of reasons. The removal of breast tissue can change the pressures on the marker allowing it to change position resulting in an “accordion effect.” Blood flow and pressure may move a marker. Post-biopsy or post-surgical mammography can cause migration of the marker. The removal of the biopsy device or other instrument may also cause a shift in the site marker due to the suction caused by a rapidly removed device. Hematoma formation and infectious processes may also cause a shift of the marker.

After surgical procedures for removing cancerous tissues, such as lumpectomies in a patient's breast, it is also may be desirable to provide a site marker in order to locate the site for further treatments such as radiation treatments to treat the cavity lining after the surgical procedures in case there may be remaining cancer cells in the cavity. Remaining cancer cells are usually found within one centimeter from the lining surface and can be successfully treated with radiation.

The movement or shift of a site marker can result in follow-up treatments being misdirected to an undesired portion of the patient's tissue. Thus devices for remotely detectable biopsy site markers that remain secured to the intended intracorporeal location are desired.

### SUMMARY OF THE INVENTION

The invention is generally directed to remotely detectable intracorporeal site markers that remain fixed at the site and allow for the subsequent accurate relocation of the site. The markers are particularly suitable for use within a cavity of a patient's breast from which tissue has been removed as in a biopsy or lumpectomy procedure

A remotely detectable marker embodying features of the invention has a tissue penetrating anchoring element and a remotely detectable marker element that is secured to the anchoring element. The anchoring element is configured to attach to the biopsy cavity wall so that the marker element is positioned within and accurately marks the cavity site. The anchoring element attaches to the biopsy cavity wall in way that resists the forces that commonly cause the migration and shifting of other less effective markers. Preferably the anchoring element has a threaded or screw-like structure or a barbed or harpoon-like construction to ensure that it does not become displaced from the tissue in which it is deployed.

The anchoring element may take alternate designs that effectively penetrate and affix marker to the biopsy cavity wall. One alternate embodiment of the anchoring element is a helical coil. Another alternate embodiment of the anchoring element includes a hook, e.g. a fish-hook, structure. Yet another alternate embodiment of the anchoring element involves a tissue penetrating anchor with an expandable component such as a molly bolt construction.

The anchoring element is designed for long term deployment within a patient's body so it should be made of biocompatible metals such as stainless steel, titanium, cobalt-chrome and other biocompatible materials. Of course for many applications the size and amounts of metal should be limited because it may interfere with remote visualization of the site. In many applications the anchoring element will need to be MRI compatible, and thus titanium may frequently be preferred. The anchoring element may also be formed of high strength biocompatible polymeric materials such as polycarbonates and polyimides.

Alternatively, for shorter term deployments, the anchoring element may be constructed of biocompatible, bioabsorbable polymeric material such as polylactic acid (PLA), polyglycolic acid (PGA), copolymers thereof, and other suitable bioabsorbable polymeric materials. Polymer-metal combinations or composites may also be employed.

A remotely detectable marker element is connected to or otherwise secured to the anchoring element. Upon the proper placement of the anchoring element in the cavity wall, the marker element is positioned in the biopsy cavity marking the location of the biopsy. The marker element provides for the subsequent remote visualization of the biopsy site via ultrasound, x-ray and/or MRI. The marker element may also serve a hemostatic function as well.

The remotely detectable marker element embodying features of the invention may have several embodiments. In one embodiment the marker element is a pellet, or string of pellets. In another embodiment the marker element is a pad or flag or cloth or braid which is at least in part formed of or has incorporated therein a metallic or other radiographically detectable element incorporated therein to provide appropriate imaging. In yet another embodiment the marker element has a one or more remotely detectable strands. For short term deployment, the marker element may be made of bioabsorbable material such as described above. The marker element may also have incorporated therein a hemostatic material such as starch or chitosan.

In one embodiment the remotely detectable marker element has tissue penetrating anchoring elements on opposing ends which are implanted into the tissue surrounding the cavity at opposing locations. This configuration allows the marker element to be placed near the center of the biopsy cavity and also assists in maintaining the shape of the body cavity.

An anchored marker embodying the features of the invention can be readily delivered to the desired location by a number of suitable delivery systems. Preferably, the delivery system has a delivery cannula that receives the marker body within an inner lumen. A plunger with a suitable handle is slidably disposed within the lumen of the delivery cannula to insert the anchoring element of the marker body into the wall of the body cavity. The leading tip of the plunger is configured to engage the head of the anchoring element in order to drive the anchoring element into the tissue wall. The tip of the plunger may have a flat tip, a Philips-type or a hex head which are configured to engage a matching recess within the head of the anchoring element.

An anchored marker embodying the features of the invention may also be configured to be inserted to the desired location through existing biopsy devices and may be designed to be inserted through both a device having a side aperture or a tip aperture.

The anchor marker embodying features of the invention is readily deployed through a suitable cannula to the desired body cavity. The anchor element is driven into the tissue wall defining the cavity by a rotating action or by pushing the element into the tissue. Once the anchor element is properly secured within the tissue wall the delivery cannula and driving plunger may be removed. Preferably, the marker element is positioned within the cavity along with the anchor element. The anchor marker is thereby securely fixed within the body cavity and is not likely to migrate. Subsequent location of the body cavity is then assured even after clotting and tissue in-growth into the cavity.

These and other advantages of the invention will become more apparent from the following detailed description of embodiments when taken in conjunction with the accompanying exemplary drawings.

### BRIEF DESCRIPTION OF THE DRAWINGS

FIG. 1 is a perspective view of an anchor marker embodying features of the invention being deployed within a cavity of a patient's breast.

FIG. 1a is an enlarged view of the cavity with the anchor element penetrating the tissue wall.

FIG. 2a is an elevational view of an anchor marker wherein the anchor element has a helical coil.

FIG. 2b is an elevational view of another anchor marker wherein the anchor element is a barbed or harpoon-like element.

FIG. 3a-3c are elevational views illustrating various marker elements of the anchor marker embodying features of the invention.

FIG. 4 is an elevational view of an embodiment of the invention in which the marker has two anchor elements which are secured to opposite sides of the cavity to maintain the position of the marker element within the body cavity.

### DETAILED DESCRIPTION OF EMBODIMENTS OF THE INVENTION

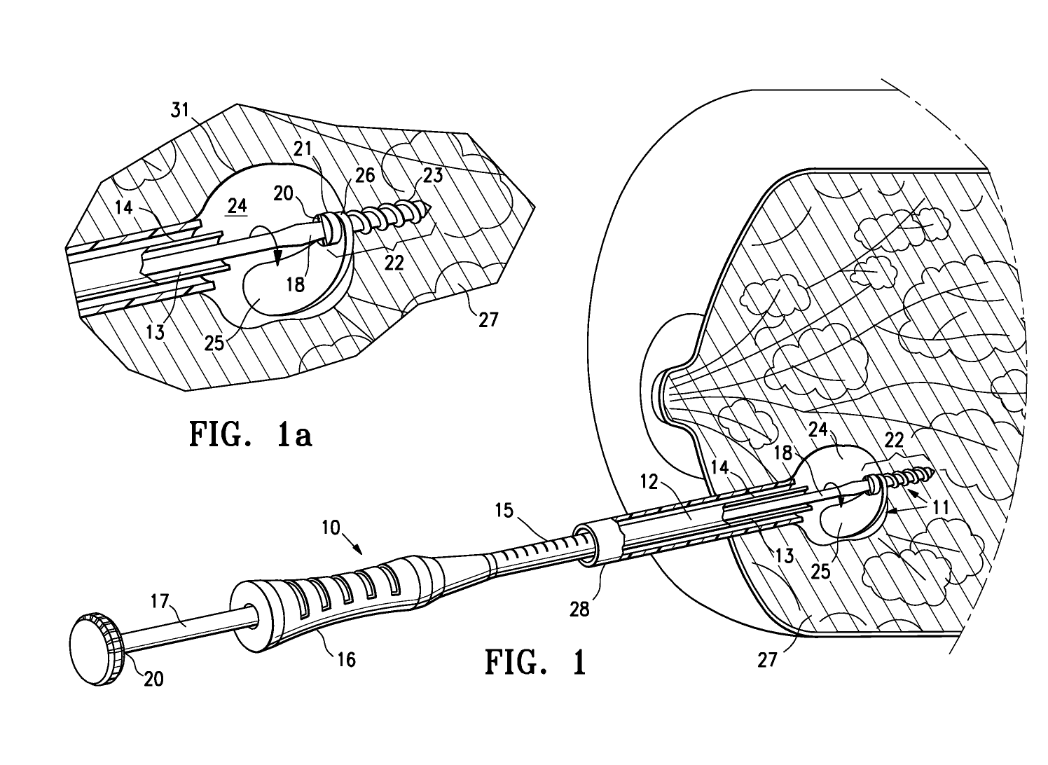

FIGS. 1 and 1a schematically illustrate a delivery system 10 for anchor marker 11 embodying the features of the invention. The delivery system 10 includes a delivery tube or cannula 12 with an inner lumen 13, a distal portion 14, and a proximal portion 15 with a handle 16. A plunger 17 is slidably disposed within the inner lumen 13 and is provided with a grip on the proximal end configured to allow an operator to advance the plunger 17. The plunger 17 has a distal tip 18 which in this embodiment is a flat screwdriver-like structure that is configured to engage a matching recess 20 in the head 21 of the anchor element 22 of the anchor marker 11. In this embodiment the anchor element 22 has a pointed and threaded shaft member 23. The plunger tip 18 is rotated and thrust forward to drive the threaded shaft member 23 of the anchor element 22 into the breast tissue 27 surrounding the cavity 24.

The remotely detectable marker element 25 of this illustrated embodiment comprises a cloth or braided material with a plurality of radiopaque strands (not shown) of stainless steel or Titanium incorporated into the cloth or braided material for subsequent imaging. The detectable marker element 25 is secured to the anchor element 22 with a collar 26 that surrounds the shaft 23 of the anchor element right below the head 21 thereof. The marker element 25 follows the anchor element 21 upon deployment and is secured within the body cavity without interfering with the deployment of the anchor element 21 into tissue 27 surrounding the cavity 24. After deployment of the anchoring element 20 into the wall of cavity 24, the remotely detectable marker element 23 resides in the cavity 24 allowing for the subsequent remote visualization of the site.

Initially, the anchor marker 11 described above is inserted into the inner lumen 13 of the delivery tube 12 proximal to the distal tip 18 of plunger 17 which is slidably disposed within the inner lumen. The delivery system is inserted into the inner lumen of an introducer cannula 28 which provides a passageway to the cavity 24 and is advanced therein until the distal end of the delivery cannula 12 extends into the cavity 24. The plunger 17 is rotated and thrust forward to engage the screw-driver like tip into the recess 20 in the head 21 of anchor element 22. The plunger is further advanced until the sharp tip of the shaft member 23 penetrates into the tissue 27 of the patient's breast. The handle of plunger 17 is further rotated until the anchor element is secured to the tissue wall. The delivery system 10 may then be removed

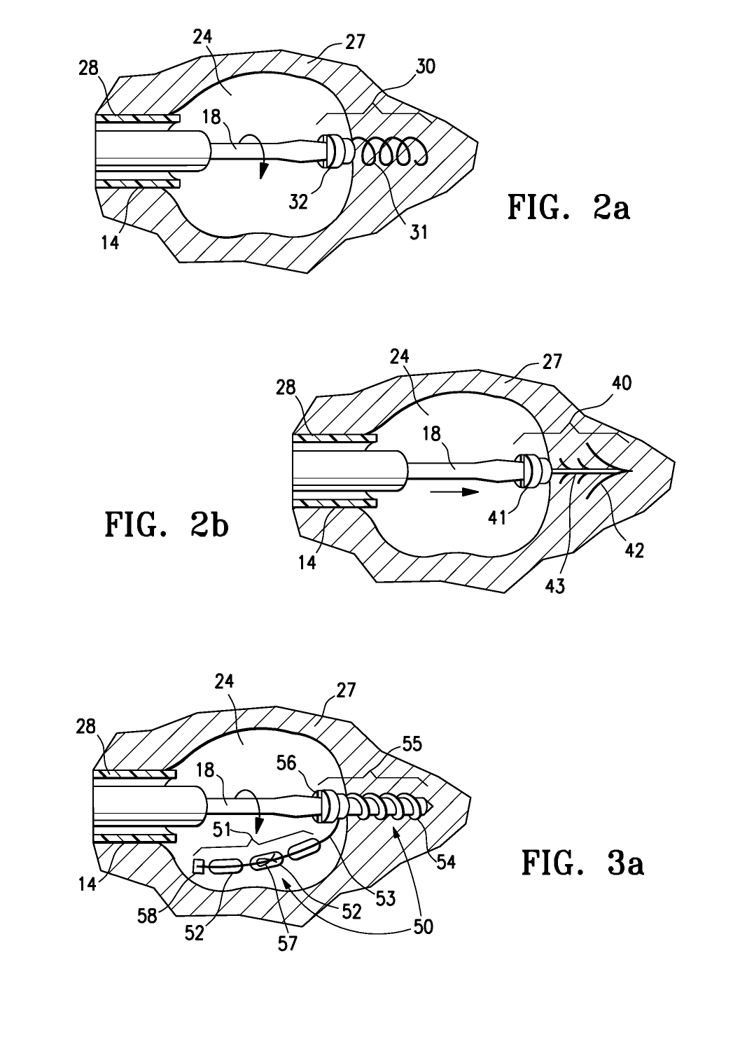

FIG. 2a illustrates an alternate embodiment of the invention wherein the anchor element 30 has a helical coil 31 with a head 32 similar to that shown in FIG. 1 for anchor element head 20. The helical coil 31 is driven into the tissue surrounding the cavity 24 in a manner similar to that shown in FIG. 1.

FIG. 2b illustrates another embodiment of the invention wherein the anchor element 40 has a head 41 and barbed elements 42 attached to tissue penetrating shaft 43. In this embodiment the anchor element 40 is pushed into the tissue wall of the cavity and the barb elements 42 hold the anchor element within the tissue wall. The anchor element 40 does not need to be rotated when deployed into the tissue wall.

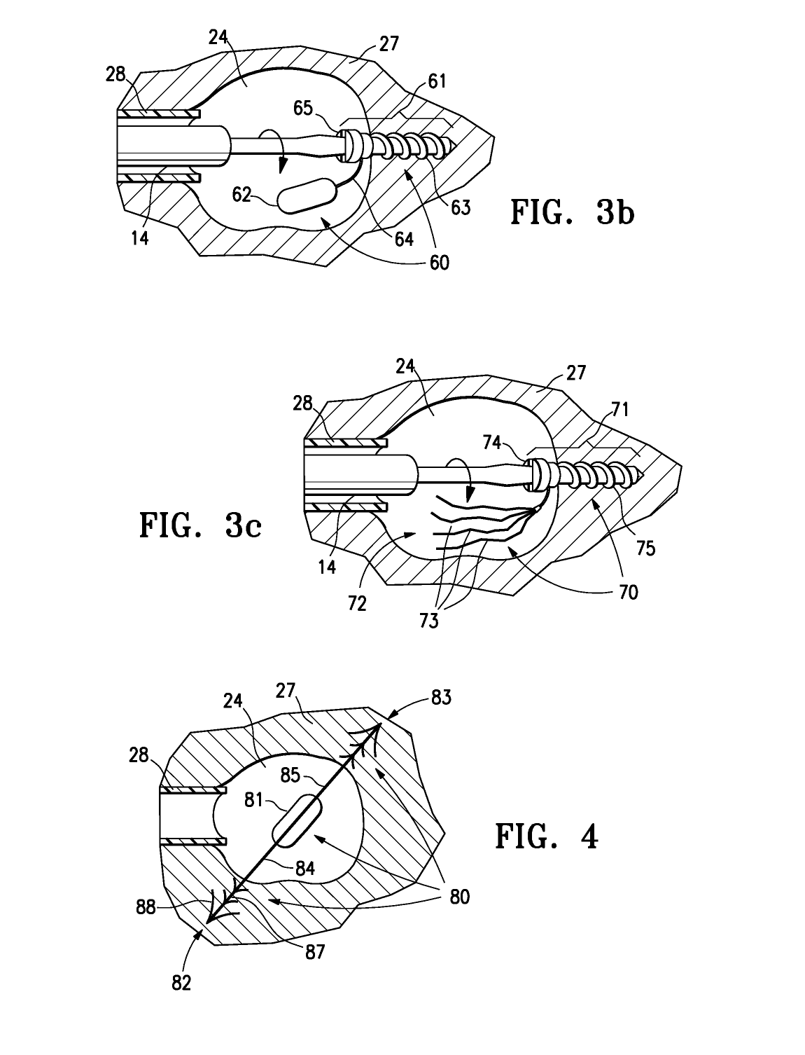

FIG. 3a illustrates yet an embodiment of the invention wherein an anchor marker 50 has a marker element 51 with a plurality of pellets 52 on a strand 53. Strand 53 has one end thereof secured to the shaft 54 of the anchor element 55 adjacent to the head 56 of the anchor element. Alternatively, each of the pellets 51 may be secured to separate strands which in turn are secured to the anchor element 55. The middle pellet has a radiopaque element 57 shaped like an alpha or gamma symbol with a loop surrounding the strand 53. If the pellets 52 are formed of bioabsorbable materials such as polylactic acid, polyglycolic acid, copolymers thereof the, radiopaque element 57 will remain on the strand and be able to mark the location of the site. The cap 58 at the free end of the strand 53 prevents the element 57 from slipping off the strand.

FIG. 3b illustrates an anchor marker 60 having an anchor element 61 and a marker element 62 that is secured to the shaft 63 of anchor element 61 by strand 64. Preferably, the end of the strand 64 is secured to the shaft 63 adjacent to the head 65. The marker element 62 may be a passive radio frequency identification (RFID) tag which allows relocation with an exterior wand which activates the RFID with RF energy so that the RFID emits a recognizable signal. The anchor element 61 secures the anchor marker 60 to the wall of cavity 24.

FIG. 3c illustrates an embodiment wherein anchor marker 70 has an anchor element 71 and a marker element 72 which has a plurality of strands 73 secured to an anchor element 70 adjacent to the head 74 of anchor element 71. The anchor element 70 is secured to the wall of cavity 24 in the same manner as the embodiments shown in FIGS. 3a and 3b wherein the shaft 75 of the anchor element is screwed in the wall of the cavity 24. The strands 73 may include radiopaque material for imaging purposes. For example one or more of the strands 73 may have or be formed of a radiopaque metallic strand (e.g. stainless steel or titanium) or the strands may have radiopaque materials such as barium sulfate incorporated therein. The strands 73 themselves may be formed of a suitable biocompatible fibrous material.

FIG. 4 illustrates an anchor marker 80 which has a marker element 81 that is connected to two anchor elements 82 and 83 by strands 84 and 85. The anchor elements 82 and 83 are deployed on opposite sides of the body cavity 24 so as to position the marker element 81 towards the center of the cavity. In this embodiment the anchor elements 82 and 83 have tissue penetrating shafts 87 with barbs 88 similar to that shown in FIG. 2b. Other types of anchor elements may be employed such as those described above in the previously described embodiments. The deployment of this anchor marker in the manner described with anchor elements on opposite sides of the cavity 24 may also help maintain the size and to a lesser extent the shape of the cavity.

While particular forms of the invention have been illustrated and described herein, it will be apparent that various modifications and improvements can be made to the invention. Additional details of the brachytherapy catheter devices may be found in the patents and applications incorporated herein. To the extent not otherwise disclosed herein, materials and structure may be of conventional design.

Moreover, individual features of embodiments of the invention may be shown in some drawings and not in others, but those skilled in the art will recognize that individual features of one embodiment of the invention can be combined with any or all the features of another embodiment. Accordingly, it is not intended that the invention be limited to the specific embodiments illustrated. It is therefore intended that this invention be defined by the scope of the appended claims as broadly as the prior art will permit.

Terms such as “element”, “member”, “component”, “device”, “means”, “portion”, “section”, “steps” and words of similar import when used herein shall not be construed as invoking the provisions of 35 U.S.C §112(6) unless the following claims expressly use the terms “means for” or “step for” followed by a particular function without reference to a specific structure or a specific action. All patents and all patent applications referred to above are hereby incorporated by reference in their entirety.

What is claimed is:

1. An anchored marker comprising:
a. at least one tissue penetrating anchor element; and
b. at least one remotely detectable marker element secured to the at least one tissue penetrating anchor element.

2. The anchored marker of claim 1 wherein the at least one tissue penetrating anchor element is a threaded element.

3. The anchored marker of claim 1 wherein the at least one tissue penetrating anchor element is a barbed element.

4. The anchored marker of claim 1 wherein the at least one tissue penetrating anchor element is a helical coil.

5. The anchored marker of claim 1 wherein the at least one tissue penetrating anchor element is an expandable element.

6. The anchored marker of claim 1 wherein the at least one marker element has a tissue penetrating anchor element at opposing positions on the marker element.

7. The anchored marker of claim 1 wherein the at least one marker element is a pellet.

8. The anchored marker of claim 1 wherein the at least one marker element is a string of pellets.

9. The anchored marker of claim 1 wherein the at least one marker element is a fibrous member.

10. The anchored marker of claim 9 wherein the at least one marker element is a cloth or braided element.

11. The anchored marker system of claim 1 wherein the at least one marker element has one or more remotely detectable strands.

12. The anchored marker system of claim 1 wherein the at least one marker element comprises at least in part a polysaccharide or starch.

13. An anchor marker delivery system, comprising:
a. an elongated tubular shaft which has a distal end, a proximal end, a discharge opening at or near the distal end, an inner lumen extending within the tubular shaft to the discharge opening;
b. at least one anchored marker slidably disposed in the inner lumen of the shaft, comprising:
i. at least one tissue penetrating anchor element; and
ii. at least one remotely detectable marker element secured to the at least one tissue penetrating anchor element; and
c. a plunger element which is slidably disposed in part within the inner lumen of the tubular shaft proximal to the at least one anchor element, and having a distal end portion configured to engage a head of the anchor element and distally advance the at least one anchor marker out of the discharge opening of the tubular shaft and to affix the tissue penetrating anchor element to or within the cavity wall.

14. The anchor marker delivery system of claim 13 wherein the distal end portion of the plunger element is a screw driver tip.

15. The anchor marker delivery system of claim 13 wherein the distal end portion of the plunger element is configured to fit within a recess within the head of the anchor element.

16. A method for delivering at least one remotely detectable marker to an intracorporeal site within a patient from which tissue has been removed or separated from surrounding tissue, comprising:
a. providing a marker delivery device comprising:
i. an elongated tubular shaft which has a distal end, a proximal end, a discharge opening in the shaft at or near the distal end, an inner lumen extending within the tubular shaft to the discharge opening, and
ii. at least one anchor marker having at least one tissue penetrating anchor element with a head and at least one remotely detectable marker element secured to the at least one tissue penetrating anchor element, and
iii. a plunger element which is slidably disposed in part within the inner lumen of the tubular shaft proximal to the at least one anchor marker, and having a distal end portion configured to engage the head of the anchor element and distally advance the at least one anchor marker out of the discharge opening and to affix the tissue penetrating anchor element to or within the biopsy cavity wall;
b. advancing the marker delivery device within the patient until the distal end of the marker delivery device is disposed at the target tissue site and the discharge opening of the marker delivery device is aligned for desired anchor marker deployment; and
c. advancing the plunger element of the marker delivery device to advance the anchor marker through the discharge opening and to affix the at least one tissue penetrating anchoring element to or within the biopsy cavity wall.
